(19) United States
(12) Patent Application Publication
(10) Pub. No.: US 2025/0312785 A1
(43) Pub. Date: Oct. 9, 2025

(54) INTEGRATED MICROFLUIDIC DEVICES WITH ISOTHERMAL AMPLIFICATION AND ELECTROCHEMICAL SENSING FOR RAPID MOLECULAR DIAGNOSTICS

(71) Applicant: Texas State University, San Marcos, TX (US)

(72) Inventor: Namwon Kim, San Marcos, TX (US)

(21) Appl. No.: 19/172,569

(22) Filed: Apr. 7, 2025

Related U.S. Application Data
(60) Provisional application No. 63/631,549, filed on Apr. 9, 2024.

(51) Int. Cl. B01L 3/00; B01L 7/00; G01N 21/64; G01N 27/30; G01N 27/327

(52) U.S. Cl. CPC B01L 3/5023; B01L 7/52; G01N 21/6428; G01N 21/645; G01N 27/301; G01N 27/308; G01N 27/3276; G01N 27/3277; B01L 2200/0647; B01L 2200/16; B01L 2300/0663; B01L 2300/0883; B01L 2300/1805

(57) ABSTRACT

An integrated sample-to-answer polymer microfluidic device that provides a rapid, accurate, and cost-effective diagnostic platform that can deliver results swiftly without sacrificing sensitivity or specificity. The microfluidic device includes a printed electrothermal heater for facilitating loop-mediated isothermal amplification (LAMP). Additionally, the microfluidic device incorporates a lateral flow membrane designed for chromatographic immunoassay and fluorescence measurement and/or a printed biosensor employed for electrochemical analysis thereby establishing a comprehensive platform for sample-to-answer detection.

### TECHNICAL FIELD

The present disclosure relates generally to molecular diagnostic techniques, and more particularly to an integrated sample-to-answer microfluidic system that integrates a polymer microfluidic device with a printed electrothermal heater for loop-mediated isothermal amplification (LAMP), a lateral flow membrane for chromatographic immunoassay and fluorescence quantification, and a printed biosensor for the electrochemical measurements.

### BACKGROUND

Molecular diagnostics combines laboratory testing with molecular biology and analyzes genomic markers for various clinical and life sciences applications, including infectious diseases, oncology, hematopathology, clinical chemistry, and clinical genetics.

There are currently many molecular diagnostic techniques to detect infectious diseases, including polymerase chain reaction (PCR), isothermal amplification reaction, gene chip technology, and high-throughput sequencing technology. PCR is the gold standard for nucleic acid amplification (rapid molecular technique that can detect small quantities of an organism's genetic material in a given specimen) due to its high sensitivity and specificity; however, it can be costly due to the need for specialized equipment. That is, existing diagnostic tests, such as PCR-based methods, offer high accuracy but are time-consuming and require laboratory settings for processing.

In contrast, antigen-based kits, while faster and more accessible, suffer from lower accuracy and sensitivity, leading to potential false negatives and contributing to the spread of infectious diseases.

Unfortunately, there is not currently a rapid, accurate, and cost-effective diagnostic platform that can deliver results swiftly without sacrificing sensitivity or specificity.

### SUMMARY

In one embodiment of the present disclosure, a polymer microfluidic device comprises a printed electrothermal heater for facilitating loop-mediated isothermal amplification. The microfluidic platform further comprises a lateral flow membrane for chromatographic immunoassay and fluorescent measurement and/or a printed biosensor employed for electrochemical analysis.

The foregoing has outlined rather generally the features and technical advantages of one or more embodiments of the present disclosure in order that the detailed description of the present disclosure that follows may be better understood. Additional features and advantages of the present disclosure will be described hereinafter which may form the subject of the claims of the present disclosure.

### BRIEF DESCRIPTION OF THE DRAWINGS

A better understanding of the present disclosure can be obtained when the following detailed description is considered in conjunction with the following drawings, in which:

FIG. 1 illustrates a schematic diagram of a chamber-type polymer microfluidic platform integrated with a printed electrothermal heater for facilitating loop-mediated isothermal amplification (LAMP) and a printed 3-electrode biosensor employed for electrochemical analysis in accordance with an embodiment of the present disclosure;

FIG. 2 illustrates a continuous flow-type microfluidic platform in accordance with an embodiment of the present disclosure;

FIG. 3A illustrates the assembly of a fabricated polymer chamber on the top of the printed silver heater in accordance with an embodiment of the present disclosure;

FIG. 3B illustrates an infrared (IR) image showing the heat confined in the area of the chamber in accordance with an embodiment of the present disclosure;

FIG. 3C illustrates the temperature profiles of heating and cooling under applied voltages of 3, 4, and 5 volts (V) along the centerline shown on the IR image of FIG. 3B in accordance with an embodiment of the present disclosure;

FIG. 3D illustrates the measured temperatures as a function of the applied voltage using inkjet and direct ink writing (DIW) printed heaters in accordance with an embodiment of the present disclosure;

FIG. 4A illustrates a printed 3-electrode electrochemical biosensor consisting of a silver electrode and graphene electrodes in accordance with an embodiment of the present disclosure;

FIG. 4B illustrates the differential pulse voltammetry (DPV) after blocking with bovine serum albumin (BSA) and hybridization of the target single-stranded DNA (ssDNA) in accordance with an embodiment of the present disclosure; and

FIG. 4C illustrates the correlation of the target ssDNA concentration with the measured peak current difference (dI) in accordance with an embodiment of the present disclosure.

### DETAILED DESCRIPTION

As stated above, molecular diagnostics combines laboratory testing with molecular biology and analyzes genomic markers for various clinical and life sciences applications, including infectious diseases, oncology, hematopathology, clinical chemistry, and clinical genetics.

There are currently many molecular diagnostic techniques to detect infectious diseases, including polymerase chain reaction (PCR), isothermal amplification reaction, gene chip technology, and high-throughput sequencing technology. PCR is the gold standard for nucleic acid amplification (rapid molecular technique that can detect small quantities of an organism's genetic material in a given specimen) due to its high sensitivity and specificity; however, it can be costly due to the need for specialized equipment. That is, existing diagnostic tests, such as PCR-based methods, offer high accuracy but are time-consuming and require laboratory settings for processing.

In contrast, antigen-based kits, while faster and more accessible, suffer from lower accuracy and sensitivity, leading to potential false negatives and contributing to the spread of infectious diseases.

Unfortunately, there is not currently a rapid, accurate, and cost-effective diagnostic platform that can deliver results swiftly without sacrificing sensitivity or specificity.

The embodiments of the present disclosure provide a means for providing a rapid, accurate, and cost-effective diagnostic platform that can deliver results swiftly without sacrificing sensitivity or specificity. The principles of the present disclosure combine the speed and ease of use comparable to antigen tests with accuracy close to PCR tests. In one embodiment, an integrated sample-to-answer microfluidic system is developed for rapid and precise molecular detection. The integrated sample-to-answer microfluidic system integrates an affordable and disposable polymer microfluidic device with a printed electrothermal heater for loop-mediated isothermal amplification (LAMP) as well as a lateral flow membrane for chromatographic immunoassay and fluorescence quantification and/or a biosensor for the electrochemical measurements using cyclic voltammetry (CV) and differential pulse voltammetry (DPV). LAMP is a highly sensitive isothermal nucleic acid amplification technique that assures high accuracy and sensitivity of test results. That is, embodiments of the microfluidic system of the present disclosure is designed for rapid and accurate molecular diagnostics that encompasses a seamless integration of an economical, single-use polymer-based microfluidic component with a printed electrothermal heater for facilitating loop-mediated isothermal amplification (LAMP). Additionally, the microfluidic system of the present disclosure incorporates either a lateral flow membrane designed for chromatographic immunoassay and fluorescence measurement or a printed biosensor employed for electrochemical analysis, establishing a comprehensive platform for sample-to-answer detection. A further discussion regarding these and other features is provided below.

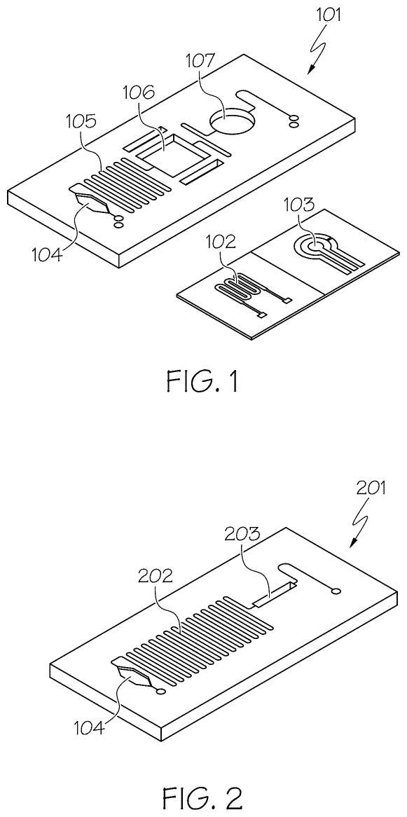

Referring now to the Figures in detail, FIG. 1 illustrates a schematic diagram of a chamber-type polymer microfluidic platform (or device) 101 integrated with a printed electrothermal heater 102 for facilitating loop-mediated isothermal amplification (LAMP) and a printed 3-electrode biosensor 103 employed for electrochemical analysis in accordance with an embodiment of the present disclosure. In one embodiment, polymer microfluidic platform 101 is a single-use polymer-based microfluidic device. It is noted that the term “platform,” as used herein, may be used interchangeably with the term “device.”

Printed electrothermal heater 102 (also referred to as a “flexible printed heater”), as used herein, is a heating device created by printing conductive and resistive inks onto a thin, flexible substrate. Loop-mediated isothermal amplification (LAMP), as used herein, refers to a rapid and specific nucleic acid amplification technique that uses a unique primer design and a strand-displacing DNA polymerase to amplify a target sequence at a single temperature eliminating the need for temperature cycling.

Printed 3-electrode biosensor 103, as used herein, refers to an electrochemical sensor consisting of three electrodes (e.g., working, reference, and counter) for detecting and quantifying biological molecules.

In one embodiment, printed 3-electrode biosensor 103 on a polymer substrate is affixed to and seals detection chamber 107 via thermal bonding or double-sided adhesive.

In one embodiment, biosensor 103 is a graphene-aptamer biosensor. In such a biosensor, the graphene's unique properties and aptamers (e.g., single-stranded DNA or RNA molecules) are used to detect specific molecules or biomarkers.

In one embodiment, biosensor 103 uses cyclic voltammetry and differential pulse voltammetry for electrochemical measurements as discussed further below. Cyclic voltammetry, as used herein, is a technique where the potential of an electrode is varied in a cyclic manner (e.g., forward and backward) and the resulting current is measured. In one embodiment, cyclic voltammetry provides information about redox processes and can be used to identify and quantify redox-active species. Differential pulse voltammetry, as used herein, is a type of voltammetry where a series of small pulses of potential are superimposed on a linear potential ramp. By measuring the current difference between the end of the pulse and the baseline, differential pulse voltammetry can enhance sensitivity and selectivity, especially for analyzing analytes with similar redox potentials.

In one embodiment, one of the three electrodes of biosensor 103 is the working electrode, which is the electrode where the electrochemical reaction occurs. In one embodiment, the working electrode is modified with a biomolecule (e.g., enzyme, antibody) to recognize and bind to the target analyte.

In one embodiment, one of the three electrodes of biosensor 103 is the reference electrode, which is the electrode which provides a stable potential reference point for the electrochemical measurements ensuring accurate and reproducible results.

In one embodiment, one of the three electrodes of biosensor 103 is the counter electrode, which is the electrode which completes the electrochemical circuit allowing the flow of current during the measurement.

In one embodiment, the three electrodes of biosensor 103 are fabricated directly onto a substrate using screen-printing, ink-jet printing, or direct ink writing (DIW) printing.

In one embodiment, chamber-type polymer microfluidic platform 101 includes a reagent chamber 104 for storing reagents. A reagent is a compound or mixture added to a system to start or test a chemical reaction. A reagent can be used to determine the presence or absence of a specific chemical substance as certain reactions are triggered by the binding of reagents to the substance or other related substances.

Furthermore, in one embodiment, microfluidic platform 101 includes a mixing channel 105 for mixing the reagent with a substance. In one embodiment, mixing channel 105 includes T- or Y-shaped junctions, serpentine or zigzag channels, and/or flow-focusing designs, which can be either passive or active.

In one embodiment, the T- or Y-shaped junctions allow fluids to mix as they flow into a common channel. In one embodiment, such mixing relies on diffusion and can be enhanced by introducing obstacles or grooves in the channel.

In one embodiment, the serpentine or zigzag channels create a tortuous flow path increasing the contact area between fluids and promoting mixing.

In one embodiment, flow-focusing designs utilize a central channel surrounded by sheath flows to focus a stream of fluid thereby creating a thin stream for efficient mixing.

In one embodiment, mixing channel 105 includes a ring mixer which operates on the principle of centrifugal forces created by the curvature of the rings, which can create counter-rotating vortices.

Additionally, in one embodiment, microfluidic platform 101 includes an amplification chamber 106 for implementing nucleic acid amplification for detecting a quantity of an organism's genetic material in a specimen consisting of the reagent mixed with the substance.

In one embodiment, amplification chamber 106 facilitates the process of nucleic acid amplification (e.g., isothermal amplification, polymerase chain reaction) to detect and measure the amount of an organism's genetic material (e.g., DNA, RNA) present in a sample.

Furthermore, in one embodiment, microfluidic platform 101 includes a detection chamber 107 for molecular detection (e.g., checking for certain changes in a gene or chromosome that may increase a person's risk of developing cancer or other diseases).

In one embodiment, detection chamber 107 employs techniques, such as polymerase chain reaction, restriction fragment length polymorphism, and fluorescence in situ hybridization, to detect mutations and changes in DNA helping assess cancer risk or diagnose genetic conditions.

As demonstrated in FIG. 1, microchannels and chambers (e.g., channels/chambers 104-107) are fabricated on microfluidic platform 101 using a micro-milling process on thermoplastic chips, including polycarbonate (PC), polymethyl methacrylate (PMMA), cyclic olefin copolymer (COC), cyclic olefin polymer (COP), etc. One or more chambers, such as chambers 106, 107, may be replaced by a continuous flow microchannel as shown in FIG. 2, which enables the application of colorimetric analysis.

Referring to FIG. 2, FIG. 2 illustrates a continuous flow-type microfluidic platform (or device) 201 in accordance with an embodiment of the present disclosure.

As shown in FIG. 2, chamber 106 of microfluidic platform 101 of FIG. 1 is replaced with a microchannel (amplification microchannel) 202 for continuous flow colorimetric analysis. In one embodiment, microchannel 202 implements nucleic acid amplification for detecting a quantity of an organism's genetic material in a specimen consisting of the reagent mixed with the substance.

In one embodiment, microchannel 202 facilitates the rapid and efficient amplification of nucleic acids (e.g., DNA, RNA) using techniques, such as polymerase chain reaction or isothermal amplification.

In one embodiment, microchannel 202 includes channels with a width and a depth between tens and hundreds of micrometers, within which fluids flow.

In one embodiment, microchannel 202 is designed to optimize the process of nucleic acid amplification by making multiple copies of a specific DNA or RNA sequence.

In one embodiment, microchannel 202 uses isothermal amplification, which amplifies nucleic acids at a constant temperature.

In one embodiment, microchannel 202 is designed to enable visible color-change detection of isothermal amplification through a transparent thin polymer coverslip.

In one embodiment, microchannel 202 is connected to detection chamber 107 (not shown in FIG. 2).

In one embodiment, detection chamber 107 of microfluidic platform 101 of FIG. 1 can also be replaced with a lateral flow assay membrane 203 for colorimetric and fluorescence analysis as shown in FIG. 2. That is, in one embodiment, detection chamber 107 of microfluidic platform 101 of FIG. 1 is replaced with lateral flow assay membrane 203 for chromatographic immunoassay and fluorescent measurement. In one embodiment, lateral flow assay membrane 203 facilitates the lateral flow of the sample and allows the analyte to interact with the immobilized components resulting in a visible signal (e.g., colored line) if the analyte is present.

In one embodiment, membrane 203 is made of nitrocellulose. In one embodiment, membrane 203 is a porous material with specific biological components (e.g., antibodies, antigens) immobilized in lines, which may appear as a thin, rectangular strip with test and control lines.

Referring to FIGS. 1-2, the formation of these microchannels (e.g., microchannel 202) and chambers (e.g., chambers 104, 106, 107) within the microfluidic platform (e.g., platforms 101, 201) can be achieved through thermoforming processes, such as hot embossing, hot pressing, and injection molding. These processes employ micromachined metal molds as mold inserts. In one embodiment, metal mold is produced through a combination of UV lithography and electroforming processes. In one embodiment, the fabricated polymer microfluidic platforms are thermally bonded using a transparent thin polymer coverslip. Such an approach enables the scalable mass production of the polymer microfluidic platforms ensuring consistent replication of microscale features essential for diagnostic applications.

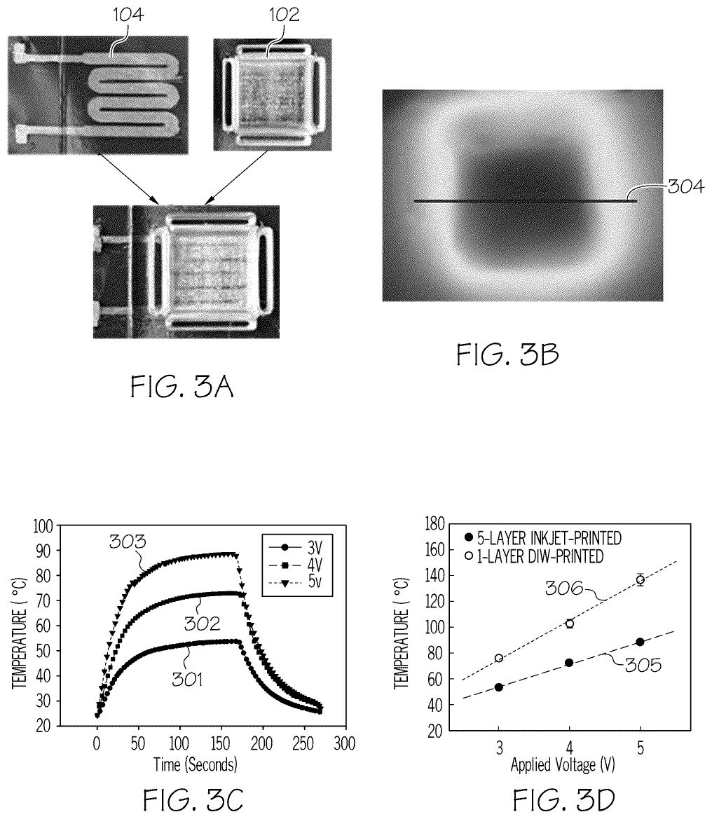

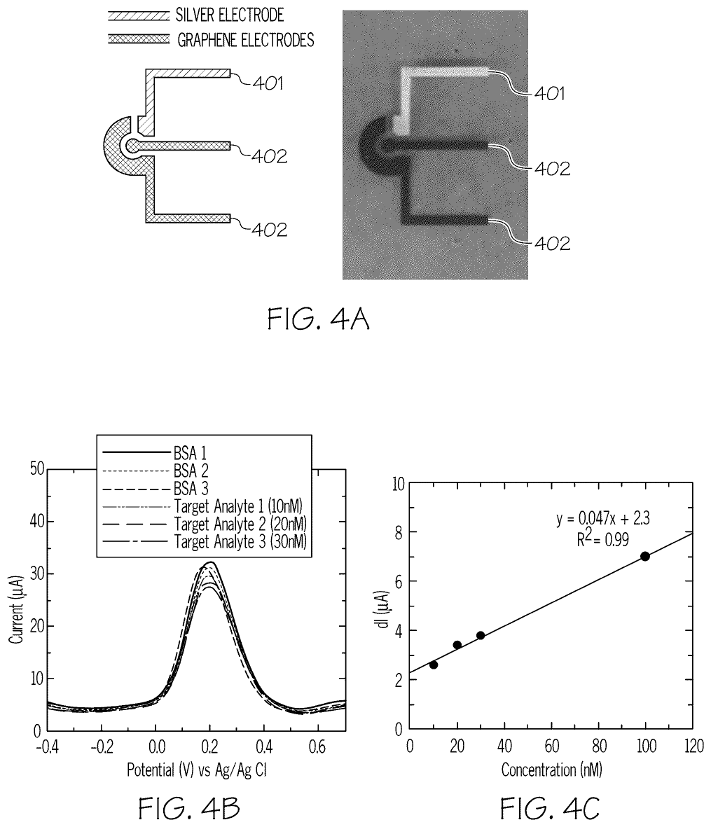

For the integration of heating and electrochemical detection functionalities, a thin film heater and a three-electrode system are fabricated on a polymer substrate using additive manufacturing techniques as shown in FIGS. 3A and 4A, including inkjet printing, direct ink writing, or aerosol jet printing. This substrate is then affixed to the microfluidic platform via thermal bonding or double-sided adhesive.

Referring to FIGS. 3A-3D, FIG. 3A illustrates the assembly of a fabricated polymer chamber (e.g., chamber 104) on the top of the printed silver heater (e.g., heater 102) in accordance with an embodiment of the present disclosure.

FIG. 3B illustrates an infrared (IR) image showing the heat confined in the area of the chamber (e.g., chamber 104) in accordance with an embodiment of the present disclosure.

FIG. 3C illustrates the temperature profiles of heating and cooling under applied voltages of 3, 4, and 5 volts (V) (see lines 301-303, respectively) along centerline 304 shown on the IR image of FIG. 3B in accordance with an embodiment of the present disclosure.

FIG. 3D illustrates the measured temperatures as a function of the applied voltage using inkjet and direct ink writing (DIW) printed heaters (see lines 305, 306, respectively, in FIG. 3D) in accordance with an embodiment of the present disclosure.

Referring now to FIGS. 4A-4C, FIG. 4A illustrates a printed 3-electrode electrochemical biosensor (e.g., sensor 103) consisting of a silver/silver chloride electrode 401 and graphene electrodes 402 in accordance with an embodiment of the present disclosure.

FIG. 4B illustrates the differential pulse voltammetry (DPV) after blocking with bovine serum albumin (BSA) and hybridization of the target single-stranded DNA (ssDNA) in accordance with an embodiment of the present disclosure.

FIG. 4C illustrates the correlation of the target ssDNA concentration with the measured peak current difference (dI) in accordance with an embodiment of the present disclosure.

Referring to FIGS. 3A-3D and FIGS. 4A-4C, in one embodiment, the heating element (e.g., heater 102), made from printed silver or graphene, generates the required heat based on the voltage applied. The effectiveness of this system is validated by measuring the temperature using an infrared camera as shown in FIG. 3B, with the setup achieving a stable temperature range between 50° to 140° C. (see FIGS. 3C and 3D), which is adequate for processes like loop-mediated isothermal amplification (LAMP). In one embodiment, the thermal system is extended for an automated temperature control mechanism by incorporating a PID temperature controller and a thermocouple.

In one embodiment, electrochemical detection is facilitated by a three-electrode system printed with graphene and silver inks as shown in FIG. 4A. The working and counter electrodes (e.g., electrodes 402) are printed with graphene ink, while the reference electrode (e.g., electrode 401) utilizes silver ink, which is subsequently modified to silver/silver chloride (see FIG. 4A). The working electrode's surface undergoes further modifications to immobilize single-strand DNA probe molecules. This modification process involves a sequence of treatments starting with oxygen plasma, followed by the application of 1-Ethyl-3-(3-dimethylaminopropyl)carbodiimide (EDC)/N-hydroxy succinimide (NHS) chemistry, and the actual probe immobilization. The subsequent bovine serum albumin (BSA) treatment to the surface prevents other molecules from any nonspecific binding on the surface, thereby enhancing the specificity of the sensor for its target analyte.

Experimental tests using complementary target single-strand DNA at various concentrations demonstrate that the printed three-electrode sensor (e.g., sensor 103), employing cyclic voltammetry (CV) and differential pulse voltammetry (DPV) techniques (see FIG. 4B), achieves a detection limit of 10 nM with a sensitivity of 0.047 μA/nM (see FIG. 4C). Such an innovative approach to microfluidic platform design enhances the capabilities for rapid, precise in vitro diagnostics, offering significant advancements over existing methods.

The principles of the present disclosure offer an integrated microfluidic system for in vitro diagnostics (IVD) that addresses several critical limitations of current diagnostic approaches, particularly for infectious diseases. In particular, the microfluidic system of the present disclosure integrates multiple functions, enabling loop-mediated isothermal amplification (LAMP) and electrochemical quantitative detection within a single polymer microfluidic architecture. Such a comprehensive integration ensures that the integrated microfluidic system can deliver rapid and accurate molecular detection by combining the speed of LAMP with the precision of electrochemical detection methods. Furthermore, the integrated microfluidic system of the present disclosure delivers quantitative results cost-effectively, making it a highly attractive solution for widespread use across various sectors. The integration of multiple diagnostic technologies into a mass-producible polymer platform not only simplifies the testing process but also significantly reduces the production time and cost, addressing one of the most pressing needs in the management of infectious diseases.

Furthermore, in one embodiment, the employment of printed biosensor 103 in microfluidic platform 101, 201 for electrochemical measurements introduces a novel approach to quantifying the concentration of target molecules.

Additionally, embodiments of the present disclosure address several critical challenges in the field of in vitro diagnostics (IVD), particularly concerning a broader range of molecular diagnostics. One of the problems addressed by the principles of the present disclosure is the need for a rapid, accurate, and cost-effective diagnostic platform that can deliver results swiftly without sacrificing sensitivity or specificity. Existing diagnostic tests, such as polymerase chain reaction (PCR)-based methods, offer high accuracy but are time-consuming and require laboratory settings for processing. In contrast, antigen-based kits, while faster and more accessible, suffer from lower accuracy and sensitivity, leading to potential false negatives and contributing to the spread of infectious diseases. The mass production of integrated polymer microfluidic systems fits into the current practice by offering a solution that combines the speed and ease of use of antigen tests with accuracy close to PCR tests. It provides a sample-to-answer platform that integrates multiple functionalities. This multifaceted approach allows for on-site testing without the need for specialized laboratory infrastructure. Potential users include healthcare providers and clinics, especially those in resource-limited settings or requiring rapid point-of-care diagnostics to make immediate clinical decisions. Public health authorities could utilize this technology for efficient disease surveillance and outbreak management, given its rapid turnaround time and high accuracy. Research and diagnostic laboratories might also find this system useful for developing and testing new diagnostic assays due to its versatility and integration of multiple detection methods.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A polymer microfluidic device, comprising:
a printed electrothermal heater for facilitating loop-mediated isothermal amplification; and
a lateral flow membrane for chromatographic immunoassay and fluorescent measurement and/or a printed biosensor employed for electrochemical analysis.

2. The polymer microfluidic device as recited in claim 1, wherein said polymer microfluidic device is a single-use polymer-based microfluidic device.

3. The polymer microfluidic device as recited in claim 1, wherein said printed biosensor is a graphene-aptamer biosensor.

4. The polymer microfluidic device as recited in claim 1, wherein said printed biosensor uses cyclic voltammetry and differential pulse voltammetry for electrochemical measurements.

5. The polymer microfluidic device as recited in claim 1 further comprises:
a reagent chamber for storing reagents.

6. The polymer microfluidic device as recited in claim 5 further comprises:
a mixing channel for mixing a reagent with a substance.

7. The polymer microfluidic device as recited in claim 6, wherein said mixing channel includes T- or Y-shaped junctions.

8. The polymer microfluidic device as recited in claim 6, wherein said mixing channel includes serpentine or zigzag channels.

9. The polymer microfluidic device as recited in claim 6, wherein said mixing channel includes a ring mixer.

10. The polymer microfluidic device as recited in claim 6 further comprises:
an amplification chamber for implementing nucleic acid amplification for detecting a quantity of an organism's genetic material in a specimen consisting of said reagent mixed with said substance.

11. The polymer microfluidic device as recited in claim 10 further comprises:
a detection chamber for molecular detection.

12. The polymer microfluidic device as recited in claim 6 further comprises:
an amplification microchannel for implementing nucleic acid amplification for detecting a quantity of an organism's genetic material in a specimen consisting of said reagent mixed with said substance.

13. The polymer microfluidic device as recited in claim 1, wherein said printed biosensor comprises a working electrode, a reference electrode, and a counter electrode.

14. The polymer microfluidic device as recited in claim 1, wherein said lateral flow membrane facilitates a lateral flow of a sample and allows an analyte to interact with immobilized components resulting in a visible signal if said analyte is present.

15. The polymer microfluidic device as recited in claim 1, wherein said lateral flow membrane is made of nitrocellulose.

16. The polymer microfluidic device as recited in claim 1, wherein said printed biosensor comprises a three-electrode system.

17. The polymer microfluidic device as recited in claim 16, wherein said three electrodes of said printed biosensor are fabricated directly onto a substrate using screen-printing, ink-jet printing, or direct ink writing (DIW) printing.

18. The polymer microfluidic device as recited in claim 16, wherein said printed biosensor comprises a silver electrode or a silver chloride electrode and two graphene electrodes.

19. The polymer microfluidic device as recited in claim 16, wherein said printed biosensor comprises electrodes printed with graphene and silver inks.

20. The polymer microfluidic device as recited in claim 19, wherein a reference electrode of said printed biosensor is printed with said silver ink, wherein working and counter electrodes of said printed biosensor are printed with said graphene ink.
